(19) United States
(12) Patent Application Publication
(10) Pub. No.: US 2023/0031527 A1
(43) Pub. Date: Feb. 2, 2023

(54) SYSTEMS AND METHODS FOR GENERATING ACCURATE OPHTHALMIC MEASUREMENTS

(71) Applicant: Alcon Inc., Fribourg (CH)

(72) Inventors: Pooria Sharif Kashani, Irvine, CA (US); George Hunter Pettit, Fort Worth, TX (US); Brant Gillen, Southlake, TX (US)

(21) Appl. No.: 17/870,304

(22) Filed: Jul. 21, 2022

Related U.S. Application Data
(60) Provisional application No. 63/227,100, filed on Jul. 29, 2021.

(51) Int. Cl. A61B 3/10

(52) U.S. Cl. CPC A61B 3/1005

(57) ABSTRACT

Certain aspects of the present disclosure provide an ophthalmic measurement device. The device comprises one or more ophthalmic measurement features, configured to generate a measurement for an anatomical characteristic of an eye of a patient, and a user interface, configured to enable a medical practitioner to interact with the ophthalmic measurement device and a memory. The device also comprises a hardware processor configured to: determine whether the measurement satisfies measurement criteria based on comparing the measurement with the measurement criteria, upon determining that the measurement does not satisfy the measurement criteria, cause the one or more ophthalmic measurement features to generate a new measurement for the anatomical characteristic, determine whether the new measurement satisfies the measurement criteria based on comparing the new measurement with the measurement criteria, and, upon determining that the new measurement satisfies the measurement criteria, cause the user interface to display the new measurement.

Aspects of the present disclosure relate to systems and methods for obtaining accurate ophthalmic measurements (e.g., pre-operative, intra-operative, etc.) for use during surgical procedures, such as cataract surgery.

Cataract surgery generally involves replacing a natural lens of a patient's eye with an artificial intraocular lens (IOL). Certain existing ophthalmic systems utilize pre-operative optical measurements of a patient's eye (for example, axial length and keratometry measurements) to help prepare a surgical plan for a cataract surgery to be performed on the patient. The surgical plan may include details for selecting an IOL type as well as an optimal IOL power, among other things, in order to achieve the desired refractive outcome. However, inaccurate measurements can lead to selecting a sub-optimal IOL power. Thus, poor quality measurements can reduce the efficacy of the cataract surgery and lead to a poor refractive outcome, which can require additional surgical or non-surgical intervention for the patient.

Therefore, there is a need for improved systems and techniques for generating accurate measurements that lead to improved refractive outcomes for patients.

### BRIEF SUMMARY

Certain embodiments provide an ophthalmic measurement device, comprising: one or more ophthalmic measurement features configured to generate a measurement for an anatomical characteristic of an eye of a patient. The ophthalmic measurement device further comprises a user interface configured to enable a medical practitioner to interact with the ophthalmic measurement device. The ophthalmic measurement device also comprises a memory and a hardware processor in data communication with the memory. The hardware processor is configured to: determine whether the measurement satisfies measurement criteria based on comparing the measurement with the measurement criteria, upon determining that the measurement does not satisfy the measurement criteria, cause the one or more ophthalmic measurement features to generate a new measurement for the anatomical characteristic; determine whether the new measurement satisfies the measurement criteria based on comparing the new measurement with the measurement criteria; and upon determining that the new measurement satisfies the measurement criteria, cause the user interface to display the new measurement.

Certain embodiments provide an ophthalmic measurement system. The system comprises an ophthalmic measurement device configured to generate a measurement for an anatomical characteristic of an eye of a patient and a user interface configured to enable a medical practitioner to interact with the ophthalmic measurement device. The system further comprises a hardware processor communicatively coupled to the ophthalmic measurement device and configured to: determine whether the measurement satisfies measurement criteria based on comparing the measurement with the measurement criteria; upon determining that the measurement does not satisfy the measurement criteria, causing the measurement device to generate a new measurement for the anatomical characteristic; determine whether the new measurement satisfies the measurement criteria based on comparing the new measurement with the measurement criteria; and upon determining that the new measurement satisfies the measurement criteria, cause the user interface to display the new measurement.

Certain embodiments provide a method for reconfiguring an ophthalmic measurement device. The method comprises aggregating a plurality of patient profiles to form a global dataset, each patient profile associated with a corresponding patient treated at one of a plurality of ophthalmic practices and comprising one or more of measurements of the anatomical characteristic of the patient's eye, procedure results, or demographics and patient history information for the corresponding patient. The method further comprises formatting each patient profile into a common format. The method also comprises identifying a first ophthalmic practice of the plurality of ophthalmic practices having a lowest average number of satisfactory results as compared to remaining ophthalmic practices of the plurality of ophthalmic practices and determining that the lowest average number of satisfactory results for the first ophthalmic practice is caused by an error associated with the ophthalmic measurement device. The method additionally comprises automatically reconfiguring the ophthalmic measurement device.

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates a block diagram of an example measurement processing system that obtains, processes, and/or verifies measurements of one or more anatomical characteristics of a patient's eye (e.g., in preparation for or during a surgical procedure), according to some embodiments described herein.

FIG. 2 is a sequence diagram illustrating operations of a server of FIG. 1 to obtain, process, and verify the accuracy of measurements for the patient's eye, according to aspects described herein.

FIG. 3 is a sequence diagram illustrating operations of a measurement device of FIG. 1 to obtain, process, and verify the accuracy of measurements for the patient's eye, according to aspects described herein.

FIG. 4 is a sequence diagram illustrating communications exchanged between or processing performed by components of the system of FIG. 1 to aggregate information from a plurality of ophthalmic practices and generate ranking information based thereon, according to some embodiments described herein.

FIG. 5 is a diagram of an embodiment of a processing system, server, or device that performs or embodies certain aspects described herein.

FIG. 6 depicts example operations for aggregating information from a plurality of ophthalmic practices and identifying one or more causes for poor refractive outcomes associated with an ophthalmic practice according to embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

As described above, in preparation for cataract surgery, a medical practitioner may use an ocular measurement device (referred to herein as a measurement device), such as an optical biometer, to obtain pre-operative measurements of one or more anatomical characteristics of the patient's eye. Examples of such anatomical characteristics include the axial length of the patient's eye, the curvature of the cornea, the lens thickness, the anterior chamber depth, and so forth. Note that a measurement herein refers to or includes a value (e.g., a number, or any other unit of measure) associated an anatomical characteristic of an eye.

Due to various reasons, in certain cases, the pre-operative measurements that are captured by the measurement device may not accurately reflect the actual measurements of the patient's eye. As such, the measurement device may provide pre-operative measurements that are inaccurate. Causes for a measurement device to output inaccurate measurements may include device-related issues (e.g., calibration issues), operator-related issues (e.g., medical practitioner performing the measurements incorrectly), and patient-related issues (e.g., patient not cooperating during the process, such as by not fixating their line of sight on a fixation point, patient is experiencing a medical condition, such as dry eye, and so forth). As described above, using inaccurate pre-operative measurements in IOL power calculations can result in the selection of an improper IOL power and, thereby, poor post-operative refractive outcomes. Certain existing pre-operative ophthalmic measurement systems and devices are, however, not equipped or configured to automatically detect inaccurate measurements.

In addition, during cataract surgery, a surgeon may utilize an intra-operative ocular measurement device, such as an intra-operative aberrometer, to verify the pre-operative measurements generated for the patient at the ophthalmic practice. For example, subsequent to removing the crystalline lens, a surgeon may use an intra-operative aberrometer to measure the curvature of the cornea and other anatomical characteristics of an aphakic eye. However, similar to pre-operative measurement devices, certain existing intra-operative ophthalmic measurement systems and devices are also not equipped and configured to automatically verify the accuracy of the intra-operative and/or pre-operative measurements.

Accordingly, certain aspects of the present disclosure provide measurement systems and devices for obtaining, processing, and verifying the accuracy of measurements associated with one or more anatomical characteristics of a patient's eye. In certain embodiments, the measurement systems and devices described herein are configured to automatically identify and flag inaccurate measurements and proactively coordinate or request re-measurement of the anatomical characteristics of the patient's eye. In certain embodiments, the measurement system and devices described herein may use new and accurate measurements to replace the inaccurate measurements for use in subsequent analysis and calculations. By replacing inaccurate measurements with accurate measurements, the medical practitioner may beneficially avoid using inaccurate measurements in subsequent analysis, determinations, IOL selections, and the like.

Some embodiments herein involve ranking ophthalmic practices that generate ocular measurements and/or perform procedures on patients' eyes based on, for example, accuracy/inaccuracy of the measurements and corresponding refractive outcomes for the procedures that utilize the measurements. Thus, different ophthalmic practices can compare their measurements, refractive outcomes, equipment, medical practitioners, and the like to identify potential areas for improvement.

Note that the systems, methods, and techniques described herein can be utilized pre-intra- and post-operatively.

### Example Measurement Processing System

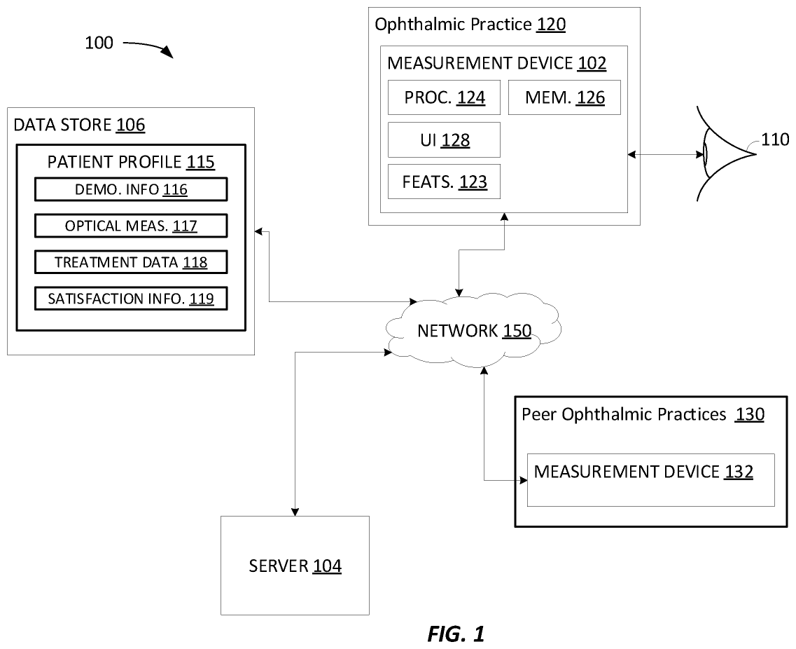

FIG. 1 illustrates a block diagram of an example measurement processing system 100 (also referred to as an ophthalmic measurement system) that obtains, processes, and verifies measurements of one or more anatomical characteristics of a patient's eye 110. The system 100 includes a server 104 that is communicatively coupled with measurement devices at various ophthalmic practices that may be located remote from each other through network 150. For example, server 104 is communicatively coupled with measurement device 102 at ophthalmic practice 120. Measurement device 102 is representative of one or more measurement devices used to measure one or more anatomical characteristics of a patient's eye 110. The server 104 is also communicatively coupled to measurement devices 132 at peer ophthalmic practices 130. In certain embodiments, measurement devices 132 comprise components similar to and function similarly to the measurement device 102. Note that an ophthalmic practice herein may refer to (1) an eye clinic at which pre-operative and/or post-operative measurements are generated for patients and/or (2) an ophthalmic surgical practice at which intra-operative measurements are generated for patients.

The server 104 is also coupled to a data store 106 that stores patient data in patient profiles 115. In certain embodiments, the data store 106 may be a central and/or cloud-based database or repository for storing patient data received from ophthalmic practice 120 and peer ophthalmic practices 130. In certain embodiments, data store 106 may be representative of an on-premise or cloud-based database or repository that is dedicated for use at a certain ophthalmic practice, such as ophthalmic practice 120.

In some embodiments, the server 104 is a central (e.g., cloud-based) computing system accessible by the ophthalmic practice 120 and the peer ophthalmic practices 130 and the corresponding measurement devices 102 and 132, respectively. For example, server 104 may correspond to computing resources (e.g., including one or more processors and/or computing systems) provided through a private or a public cloud. In certain embodiments, server 104 may refer to a computing system that is dedicated and/or local to ophthalmic practice 120. In certain embodiments, the network 150 may include one or more switching devices, routers, local area networks (e.g., an Ethernet), wide area networks (e.g., the Internet), and/or the like.

The measurement device 102, as shown in FIG. 1, comprises any ocular measurement device configured to generate measurements for one or more of the curvature and astigmatism of the front corneal surface, the axial length, the anterior chamber depth, the central corneal thickness, corneal diameter, a lens thickness, anterior corneal shape, and any other measurements associated with various other optical components of the patient's eye 110. In some embodiments, the measurement device 102 comprises one or more of a keratometer, an optical biometry device, an autorefractometer, a corneal topographer, an ocular wavefront aberrometer, an optical coherence tomography (OCT) device, an ophthalmometer, an intra-operative OCT device, swept source OCT device, intra-operative aberrometry device, and the like.

Measurement device 102 includes a processor 124 that, in some embodiments, executes instructions provided by memory 126 to generate and process measurements, process sensory data (e.g., provided by device features 123) to generate measurements, generate and process image data, verify measurements, cause measurements to be displayed, allow an operator to operate measurement device 102 through user interface 128, etc. The measurement device 102 also includes the memory 126, which may correspond to a local storage (e.g., volatile or non-volatile) for storing instructions and/or data used by the processor 124 for processing and analysis. Further details of the analysis by the processor 124 are provided below.

The measurement device 102 further includes a user interface 128 that enables a user, such as a medical practitioner, to interact with and control the measurement device 102. The user interface 128 comprises any interface through which the medical practitioner can manipulate, interact with, or view data, such as patient profile data, measurements, equipment parameters, and the like. In some embodiments, the user interface 128 comprises a graphical user interface through which the medical practitioner can manipulate, interact with, and operate the measurement device 102.

The measurement device 102 includes device features 123 for measuring the one or more anatomical characteristics of the patient's eye 110 and generating measurements based thereon. Non-limiting examples of device features 123 include at least one of optical features, emission features, sensor/imaging features, and control features. The optical features comprise one or more lenses or other optical components for focusing and directing light projected to and reflected by a target object of the patient's eye 110. The optical features enable the measurement device 102 to view and analyze the patient's eye 110, focus optical beams into the eye, etc., to generate measurements for the patient's eye 110.

The emission features comprise a light or other signal source configured to project a signal (e.g., optical beam, ultrasonic sound waves, etc.) into the patient's eye 110. The emission features may be adjustable with regard to positioning, focusing, power level, or otherwise directing the signal as needed by the medical practitioner or in an automated manner. The sensor/imaging features include features that generate, receive, process, and/or digitize signals that that reflect or echo back from the eye. The sensor/imaging features are responsible for generating multi-dimensional images and/or measurements based on the received signals. The sensor/imaging features may acquire, store, and/or process image data based on the received signals. Examples of sensor/imaging features in an OCT device may include photodetectors, digital signal processing components, image processing components, etc.

The control features enable the medical practitioner to activate, deactivate, and adjust the device features 123 of the measurement device 102. For example, the control features include components that enable adjustment of the emission features, such as controls to turn on/off the emission features, and so forth. Similarly, the control features include components that enable adjustment of the optical features, such as to enable automatic or manual focusing of the optical features or movement of the optical features to view different targets or portions of the target. In some embodiments, the user interface 128 includes the control features.

In certain embodiments, the ophthalmic practice 120 may use the measurement device 102 to obtain and process pre-operative measurements to prepare a surgical plan in preparation for a surgical procedure (e.g., cataract surgery). In certain embodiments, the ophthalmic practice 120 may use the measurement device 102 in connection with an operating room to obtain and process intra-operative measurements prior to completion of the surgical procedure.

The measurement device 102 communicates measurements to the server 104 for processing and storage. The server 104 comprises one or more processors and corresponding memory (not shown) that manage access to the data store 106 and process data accessible via the network 150. As part of the processing and storage, the server 104 may receive the measurements from the measurement device 102 and associate and store the measurements with the patient profile 115 for the patient whose eye 110 was measured.

As described above, the data store 106 stores patient profiles 115 of patients for whom measurements are generated at the ophthalmic practice 120 or peer ophthalmic practices 130. Each patient profile 115 in the data store 106 may store the patient's historical and demographic information 116, optical measurements 117, actual treatment data 118 associated with the patient's surgery, and patient satisfaction information 119 for the corresponding patient. In some embodiments, the patient profile 115 further includes information about the ophthalmic practice 120 at which measurements were taken or a procedure was performed and about the measurement device 102 that was used to generate measurements for the patient's eye 110.

The historical and demographic information 116 for each patient includes patient age, sex, ethnicity, race, prior surgery information, underlying conditions (for example, eye diseases), genetic makeup, patient lifestyle (for example, use of digital display screens for long periods of time), and the like. The optical measurements 117 may include pre-operative, intra-operative, and/or post-operative measurements, provided by one or more measurement devices, such as the measurement device 102. In some embodiments, the optical measurements 117 include other details of anatomical characteristics of the patient's eye(s), as would be known to one of ordinary skill in the art. In some embodiments, the optical measurements 117 may include flag data to indicate one or more flags for optical measurements stored therein, such as an accurate or inaccurate measurement flag for pre- or intra-operative measurements. The accurate measurement flag indicates an accurate measurement, while the inaccurate measurement flag indicates an inaccurate measurement.

Accurate measurements, as used herein, correspond to measurements generated by the measurement device 102 that have an expected or desired relationship with measurement criteria, described below, for the anatomical characteristic of the patient's eye 110. On the other hand, inaccurate measurements correspond to measurements that do not have the expected or desired relationship with the measurement criteria for the anatomical characteristic of the patient's eye 110. Examples of different measurement criteria are provided below.

The actual treatment data 118, for example, includes the actual IOL parameters (IOL type, IOL power, etc.) of the IOL used for the patient, as well as any additional relevant information relating to the treatment of the patient. The actual treatment data 118 may indicate the method of performing the cataract surgery for the patient, the tools used for the treatment, and other information about specific procedures performed during the surgery. In some embodiments, the actual treatment data 118 includes information regarding the medical practitioner that performed the surgery or generated the surgical plan or information regarding the medical equipment used before and during the surgery. The patient satisfaction information 119 included in each patient profile 115 may indicate the patient's satisfaction with the treatment as a binary indication of satisfaction or dissatisfaction with the results of the surgery.

The data store 106 further stores measurement criteria for verifying the accuracy of the measurements provided for a patient's eye 110 (e.g., left eye). The measurement criteria may include (1) measurements associated with the patient's other eye (e.g., right eye), and a threshold distance corresponding to the expected range of difference between measurements associated with the patient's left and right eye, (2) previously generated measurements associated with the same eye, i.e., patient's eye 110, and a threshold distance corresponding to the expected range of difference between the currently generated measurements and the previously generated measurements associated with the same eye, (3) one or more threshold ranges to determine whether respective measurements are outside the range of normal or typical measurements, and the like. Note that measurement criteria may refer to or include a single measurement criterion or multiple measurement criteria.

Further, a measurement criterion may be patient-specific or non-patient-specific. Patient-specific measurement criteria may refer to information that is defined or determined for the patient, for example, based on the patient's information stored in patient profile 115. Non-patient-specific measurement criteria may refer to information that is used generally for all patients. In one example, patient-specific measurement criteria is stored in the patient profile 115 as part of the optical measurements 117. Non-patient-specific measurement criteria may be stored as part of the patient's profile 115 or in the data store 106 with broader applicability. In some embodiments, the different types of measurement criteria are applied in a particular order or priority. For example, the patient-specific measurement criterion that includes measurements associated with the patient's other eye may be prioritized over other measurement criteria.

In an example, the measurement device 102 generates a measurement for the axial length of a patient's left eye, and the measurement criteria used to verify whether the left eye's axial length measurement is accurate includes (1) the axial length of the patient's right eye and (2) a threshold distance. While the axial length of the patient's right eye is patient-specific, in certain embodiments, the threshold distance can be patient-specific or non-patient-specific.

The threshold distance, in combination with the axial length of the patient's right eye, identifies an expected range in which an accurate axial length measurement for the patient's left eye is expected to fall. For example, if the axial length of the patient's right eye is 22 millimeters (mms) and the threshold distance is 0.5 mms, then an axial length of 22.3 mm that is generated by measurement device 102 for the patient's left eye may be deemed accurate (i.e., 22.3-22<0.5). However, in that example, an axial length of 23 mm that is generated by measurement device 102 for the patient's left eye may be deemed inaccurate (i.e., 23-22 >0.5).

A non-patient-specific threshold distance may be based on observations of the differences between measurements (e.g., axial length, curvature of the cornea, etc.) of the right and the left eyes associated with a large number of patients (e.g., thousands or millions of patients). For example, a non-patient-specific threshold distance may correspond to an average difference between right and left eye measurements in a pool of patients. On the other hand, a patient specific threshold distance refers to a threshold distance that is specifically determined for the patient. Applying a patient-specific threshold distance may be particularly advantageous because correlations between a patient's left and right eyes may be different depending on the historical and demographic characteristics of the patient.

For example, the difference between the axial lengths of the left and rights eyes of patients with a first characteristic (e.g., type of race or ethnicity, prior surgery, genetic makeup, underlying condition) may go up to 0.7 mms while the difference between the axial lengths of the left and rights eyes of patients with a second characteristic (e.g., type of race or ethnicity, prior surgery, genetic makeup, underlying condition) may only go up to 0.5 mms. In such an example, when comparing measurements of left and right eyes, a threshold distance of 0.7 mms may be more appropriate to use for a patient with the first characteristic while a threshold distance of 0.5 mms may be more appropriate to use for a patient with the second characteristic. This is a very simplified example of why it may be advantageous to use a patient-specific threshold distance (or other measurement criteria) when verifying a patient's measurements. In some embodiments, the patient-specific threshold distance can be determined through use of machine learning, as further described below.

Alternatively, the patient-specific threshold distance can be identified using a rule-based approach in combination with a threshold distance library. In such an example, the threshold distance library may include different threshold distances for different types of patient populations. These different types of patient populations may be categorized based on their demographic information, underlying conditions (e.g., eye diseases), genetic make-up, prior procedures (such as a prior cataract surgery or laser-assisted in-situ keratomileusis (LASIK) surgery), etc. For example, a threshold distance used for a patient with no eye conditions may be different from a patient whose one is highly myopic compared to the other. In such an example, a larger difference between the patient's axial lengths may be determined to be acceptable and not necessarily indicative of inaccurate measurements. Therefore, using a rule-based model, a first population with a first eye condition background, for instance, has a threshold distance different from a second population with a second eye condition background. As a result, in a rule-based approach, what threshold distance is used to verify the accuracy of a patient's measurements values would then depend on what population into which the patient falls.

As described above, in another example, the measurement criteria may include previously generated measurements of the same eye, i.e., patient's eye 110, and a threshold distance corresponding to the expected range of difference between the currently generated measurements and the previously generated measurements associated with the same eye. A currently generated measurement refers to a measurement whose accuracy is being verified. Because an eye's anatomical characteristics are not expected to change much (at least over a short period of time and assuming the eye has not experience trauma, surgery, disease, etc.), comparing a currently generated measurement for an eye with a previously generated measurement for the same eye may be indicative of whether the currently generated measurement is accurate. A threshold value may also be used in this comparison. For example, the corneal curvature is not expected to change by more than a certain percentage, such as 5% or so, over 70-80 years, in which case if the currently measured corneal curvature is within 5% of the previously measured corneal curvature, then the currently measured corneal curvature would be deemed accurate. A threshold distance used for comparing a currently generated measurement and a previously generated measurement may also be patient-specific (e.g., determined using a rule-based approach, machine learning, etc.) or non-patient-specific.

As described above, a patient-specific threshold distance (e.g., used for comparison between measurements of the different eye or for comparison between a currently generated measurement and a previously generated measurement) may be determined through use of machine learning, as further described below. For example, the server 104 may use a trained ML model to recommend a threshold distance for a patient based on the patient's specific information stored in patient profile 115. The patient profiles 115 may provide records of patients to generate a dataset (referred to as the “training dataset”) for use in training the ML model that can recommend patient-specific threshold distances for use in verifying accuracy of measurements.

In some instances, the server 104 may employ a model trainer used to train the ML model. The model trainer uses one or more ML algorithms in conjunction with the training dataset to train the ML model. In certain embodiments, a trained ML model refers to a function, for example, with weights and parameters, that is used to generate or predict a patient-specific threshold distance for a given patient. In some embodiments, different ML algorithms may be used to generate different threshold ranges, and the like, for the patient. For example, one model may be trained to recommend a threshold distance for verifying the patient's axial length measurement and another model may be trained to recommend a threshold distance for verifying the patient's corneal curvature measurements.

The ML algorithms may include a supervised learning algorithm, an unsupervised learning algorithm, and/or a semi-supervised learning algorithm. Unsupervised learning is a type of machine learning algorithm used to draw inferences from datasets consisting of input data without labeled responses. Supervised learning is the ML task of learning a function that, for example, maps an input to an output based on example input-output pairs. Supervised learning algorithms, generally, include regression algorithms, classification algorithms, decision trees, neural networks, etc.

Once trained and deployed, based on a certain set of inputs, including the patient's information, the ML models are able to generate or predict a threshold distance that is specific for the patient, as output. In certain aspects, the model trainer trains a multi-input-single-output (MISO) ML model that is configured to take a set of inputs associated with the patient and provide a threshold distance that is specific for the patient. To train the MISO ML model, in some embodiments, the model trainer may utilize a labeled dataset generated based on patient profiles 115 of a large number of patients. The dataset, in such embodiments, includes a plurality of samples, each indicating, for example, historical and demographic information, optical measurements, actual treatment data, and patient satisfaction information for a certain historical patient.

For example, each sample in such a dataset includes i) input data from a patient profile 115 including one or more of a patient's historical and demographic, optical measurements, actual treatment data, etc.; ii) output data including the threshold distance used to verify a measurement for the patient, and iii) patient satisfaction information. To train the MISO ML model, model the trainer runs each sample through the MISO ML model to predict a threshold distance that would hypothetically result in identifying accurate measurements that result in satisfactory surgical results. The model trainer then trains the MISO ML model based on the resulting error (i.e., Y-Y\hat{ }), which refers to a difference between the threshold distance predicted by the MISO ML model and the actual threshold distance used for the corresponding patient, as indicated in the patient record.

In other words, the model trainer adjusts the weights in the ML model to minimize an error (or divergence) between the predicted threshold distance and the threshold distance used for verifying the measurements for a patient that indicated a satisfactory surgical result. By running many more samples through the MISO ML model and continuing to adjust the weights, after a certain point, the MISO ML model starts making very accurate predictions with a very low error rate. At that point, the MISO ML model is ready to be deployed for taking a set of inputs about a current patient and predicting an optimal threshold distance that would increase the likelihood of a satisfactory surgical outcome for the current patient. The trained MISO model may be deployed for use by the server 104 or processor 124 for verifying measurements for the current patient. The recommended threshold distance can then be stored in the patient profile 115 in the data store 106.

Note that, in some embodiments, the measurement criteria, for example, stored in the data store 106 can be updated. New patient-specific or non-patient-specific measurement criteria can be generated and stored in the data store 106 as new measurement criteria or replacing existing measurement criteria.

In certain embodiments, server 104 verifies the accuracy of measurements that are generated by measurement device 102. For example, the server 104 compares measurements that are generated by the measurement device 102 and transmitted to the server 104 over network 150 to measurement criteria stored in the patient profile 115 in the data store 106 to determine whether the measurements are accurate, as further described below.

In certain other embodiments, processor 124 of the measurement device 102 verifies the accuracy of measurements that are generated by the measurement device 102, or device features 123 thereof. For example, the processor 124 compares the measurements that are generated by the measurement device 102 to measurement criteria obtained from the patient profile 115 transmitted over the network 150 from the data store 106 to the processor 124.

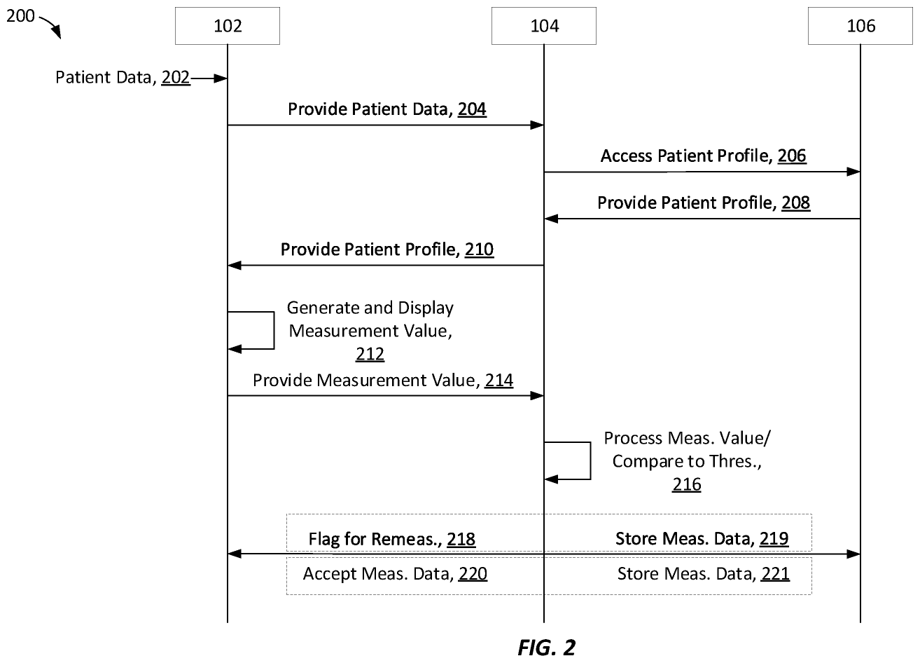

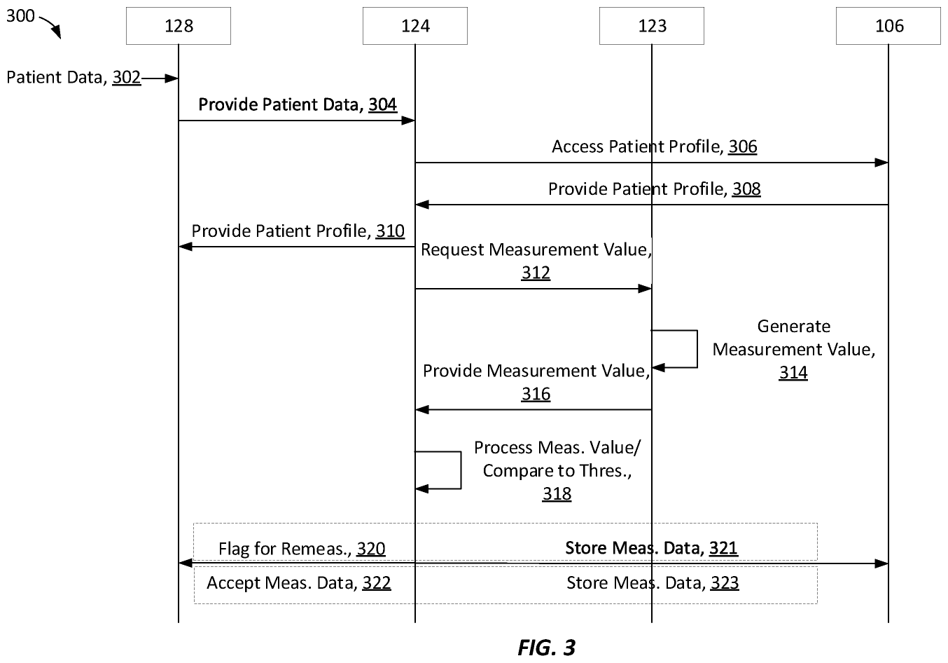

FIG. 2 below describes a sequence diagram in which the server 104 verifies the accuracy of measurements, for example, in a cloud-based system including various components of FIG. 1. On the other hand, FIG. 3 below describes a sequence diagram in which the processor 124 verifies the accuracy of measurements that are generated by the measurement device 102.

FIG. 2 is a sequence diagram 200 illustrating communications exchanged between or processing by components of the system 100 of FIG. 1 in, for example, a cloud-based architecture to obtain, verify, and process measurements for anatomical characteristics of a patient's eye (e.g., patient's eye 110), according to aspects described herein. While the sequence diagram 200 and corresponding description include reference to components of the system 100 of FIG. 1, the steps of the sequence diagram 200 are not limited to that example embodiment and may apply to various other combinations of components. Furthermore, the sequence diagram 200 is not required to perform each of or only the shown steps and is not limited to performing the indicated steps in any particular order.

The sequence diagram 200 depicts interactions between the server 104, the data store 106, and the measurement device 102. The sequence diagram 200 begins at communication step 202 with measurement device 102 receiving patient identification data through, for example, user input. The patient identification data, which may comprise the patient's name, identifier, or the like, identifies the patient whose eye 110 a medical practitioner is measuring with the measurement device 102. In some embodiments, the medical practitioner provides the patient identification data to the measurement device 102 via, for example, the user interface 128. Alternatively, a user interface at the ophthalmic practice 120 separate from the user interface 128 receives the patient identification data and provides it to the measurement device 102 or to the server 104.

At communication step 204, the measurement device 102 communicates the patient identification data received at step 202 to the server 104.

At communication step 206, the server 104 uses the patient identification data received in the step 202 to access the patient profile 115 in the data store 106 for the corresponding patient. Alternatively, the server 104 may use the patient identification data to query the data store 106 to provide the patient profile 115 for the corresponding patient.

At communication step 208, the data store 106 provides the server 104 with the requested patient profile 115 and corresponding patient-specific and non-patient-specific measurement criteria. As described further below, the server 104 can use the measurement criteria to verify the accuracy of measurements generated by the measurement device 102 at step 212.

At communication step 210, the server 104 optionally provides the patient profile 115 to the measurement device 102.

At processing step 212, the measurement device 102 generates measurements (e.g., axial length of the eye, curvature of the cornea, etc.) for the anatomical characteristic of the patient's eye, for example, using the device features 123 of the measurement device 102. The measurement device may also display the measurements (e.g., in the form of images, values, 3D models, etc.) for review by the medical practitioner, for example, on the user interface 128 of the measurement device 102 or the like to enable the medical practitioner to identify any concerns with the measurements.

At communication step 214, the measurement device 102 provides the measurements to the server 104.

At processing step 216, the server 104 processes at least one of the measurements to determine whether the measurement is accurate or inaccurate. As introduced above, the server 104 may verify the accuracy of the measurement based on comparing the measurement to measurement criteria.

For example, the server 104 receives at step 214, a measurement for the axial length of one of the patient's eyes (referred to as the first eye's measurement) from the measurement device 102. The server 104 may also receive measurement criteria including previously obtained measurements for the axial length of the patient's other eye (referred to as the second or the other eye's measurement) and a threshold distance, for example, from the patient profile 115. Note that, in one example, the second eye's measurement may be obtained as part of the measurements received from measurement device 102 at step 212. In another example, the second eye's measurement may be received as part of the optical measurements 117 that are obtained by the server 104 when the server 104 receives the patient profile at step 208.

Comparing the first eye's measurement with the second eye's measurement to determine whether the first eye's measurement is accurate or inaccurate may comprise the server 104 calculating a difference between the first eye's measurement and the second eye's measurement. The server 104 then compares the difference to the threshold distance. Where the difference between the first eye's measurement and the second eye's measurement is within the threshold distance, the server 104 identifies the first eye's measurement as accurate; where the difference is greater than the threshold distance, the server 104 identifies the first eye's measurement as inaccurate. As such, the server 104 is able to determine accuracy of the first eye's measurement based on measurement criteria including the second eye's measurement and the threshold distance.

In certain embodiments, the server 104 may compare the currently generated measurement (i.e., the measurement whose accuracy is being verified by the server 104) to a previously generated measurement for the same eye. For example, the currently generated measurement corresponding to the axial length of the right eye may be compared with a previously generated measurement corresponding to the axial length of the same eye to calculate a difference. The server 104 then compares the difference to the threshold distance. Where the difference between the currently generated measurement and the previously generated measurement is within the threshold distance, the server 104 identifies the currently generated measurement as accurate; where the difference is greater than the threshold distance, the server 104 identifies the currently generated measurement as inaccurate.

In certain embodiments, a previously generated measurement may include a measurement generated for the patient for a previous surgery. For example, measurement device 102 may be a pre-operative measurement device that has generated a measurement associated with the curvature of the cornea for a patient's eye in preparation for cataract surgery. To determine whether the pre-operative measurement is accurate, the server 104 may compare the currently generated measurement with a measurement that was generated prior to the patient's previous surgery. Such a comparison may provide a good indication of whether the pre-operative measurement is accurate if the previous surgery is not the type of surgery that would impact measurements of the eye's optical components. However, in cases like laser-assisted in-situ keratomileusis (LASIK) surgery, the measurements of the patient's eye may be heavily impacted by the surgery. In such cases, the server 104 may instead compare the currently generated measurement with a measurement that was generated after the patient's LASIK surgery.

In certain embodiments, a previously generated measurement may include a pre-operative measurement generated for the patient by a measurement device other than measurement device 102 (measurement generated at the same clinic and the same day but with a different measurement device (e.g., another manufacturer, etc.)). For example, measurement device 102 may be pre-operative measurement device that has generated a measurement associated with the curvature of the cornea for a patient's eye in preparation for cataract surgery. To determine whether the pre-operative measurement is accurate, the server 104 may compare the currently generated measurement with a measurement that was generated by another pre-operative measurement device.

In certain embodiments, a previously generated measurement may include a pre-operative measurement generated for the same surgery. For example, measurement device 102 may be an intra-operative measurement device that has generated a measurement associated with the curvature of the cornea for a patient's eye. To determine whether the intra-operative measurement is accurate, the server 104 may compare the intra-operative measurement (e.g., currently generated measurement) with a pre-operative measurement (e.g., previously generated measurement) provided by a clinic.

In certain embodiments, a previously generated measurement may include an intra-operative measurement generated by a device other than measurement device 102. For example, measurement device 102 may be an intra-operative measurement device that has generated a measurement associated with the curvature of the cornea for a patient's eye. To determine whether the intra-operative measurement is accurate, the server 104 may compare the intra-operative measurement (e.g., currently generated measurement) with an intra-operative measurement (e.g., previously generated measurement) provided by another intra-operative measurement device, e.g., at the same surgical facility on the same day.

As described above, the threshold distance (e.g., whether used for comparison between measurements of the right and the left eyes or for comparison between a currently generated measurement and a previously generated measurement) may be patient-specific or non-patient specific. In examples where the threshold distance is patient-specific, the server 104 may use (1) a ML model to determine a threshold distance that is specific to the patient based on the patient's own information in the patient profile 115, or (2) a threshold distance library based on what population the patient falls into, as described above.

In certain embodiments, the server 104 may compare the current measurement from the measurement device 102 to a threshold range to determine whether the current measurement for the patient's eye 110 is accurate. If the current measurement falls inside the threshold range, then the server 104 identifies that the current measurement is accurate. If the current measurement falls outside the threshold range, then the server 104 identifies that the current measurement is inaccurate. As an example, if the axial length of a human eye generally falls in the range of 18 mm to 27 mm, then a measurement that indicates a 60 mm axial length measurement may be indicative of an inaccurate measurement. In some embodiments, other examples of measurements that can be verified for accuracy based on comparison of corresponding measurements between the patient's eyes include anterior chamber depth measurements, lens thickness measurements, and cornea thickness measurements, among others.

In certain embodiments, if the server 104 detects a pattern of multiple measurements that are generated by measurement device 102 for different patients and that fall outside of corresponding threshold ranges, then the server 104 may determine that the measurement device 102 is out of calibration, as described in further detail below. In such embodiments, the server 104 may (1) automatically cause the measurement device 102 to display a prompt indicating that measurement device 102 is out of calibration, (2) automatically calibrate the measurement device 102, (3) automatically notify maintenance technicians to evaluate and calibrate the measurement device 102, or (4) terminate or cause the operations of the measurement device 102 to be terminated.

As introduced above, the server 104 may select the individual criterion from the measurement criteria in a particular order or priority. In some embodiments, the server 104 may select to verify the accuracy of the measurement by comparing the measurement to multiple measurement criteria. In some embodiments, the server 104 may select the measurement criterion for use in verifying the accuracy of the measurement based on the measured anatomical characteristic, where measurements for particular anatomical characteristics employ specific measurement criterion to verify accuracy.

If, at step 216, the server 104 determines that a measurement generated by the measurement device 102 is inaccurate, then at communication step 218, the server 104 may flag the measurement as inaccurate and request that the measurement device 102 re-measure the patient's eye. In some embodiments, the measurement device 102 indicates the inaccurate measurement and the re-measurement request to the medical practitioner, for example, via the user interface 128. In some embodiments, the server 104 causes the user interface of the measurement device 102 to display the inaccurate measurement, how inaccurate the inaccurate measurement is (for example, the difference between the inaccurate measurement and the measurement criterion), a recommended course of action to correct the inaccurate measurement based on identifying a cause for the inaccurate measurements. Though not shown, the medical practitioner may re-measure the patient's eye with the measurement device 102 or override the request to re-measure. Note that, in some embodiments, after determining that the measurement is inaccurate, the server 104 may automatically cause the measurement device 102 to re-measure the patient's eye without any input from the medical practitioner.

Note that although certain embodiments herein are described with respect to verifying the accuracy of an axial length measurement of a patient's eye, as described above, the embodiments described herein are similarly applicable to verifying the accuracy of other measurements, such as a keratometry measurement (e.g., average K), an anterior chamber depth measurement, lens thickness measurement, a cornea thickness measurement, and so forth of a patient's eye.

At communication step 219, the server 104 may store the inaccurate measurement and the corresponding flag in the patient profile 115, as introduced above. In some embodiments, the server 104 may also store information such as the difference between the inaccurate measurement and the measurement criterion, a recommended course of action to correct the inaccurate measurement, and the like in the patient profile 115.

If, at step 216, the server 104 determines that the measurement is accurate, then at communication step 220, the server 104 indicates to the medical practitioner, for example via the user interface 128, that the measurement is accurate. At communication step 221, the server 104 then stores the accurate measurement in the patient profile 115 in the data store 106 for future access.

Note that in sequence diagram 200, the server 104 performs either steps 218 and 219 or steps 220 and 221 but not both. In other words, if the measurement is inaccurate, then steps 218 and 219 may be performed, and if the measurement is accurate, then steps 220 and 221 may be performed. Also, though not shown in the sequence diagram 200, re-measurement of the patient's eye and verifying the accuracy of the measurements generated as a result of the re-measurement, may comprise repeating steps 212-216 as well as 218-219 or 220-221.

In some embodiments, the server 104 monitors information for the measurement device(s) (such as the measurement device 102) at particular ophthalmic practices (such as the ophthalmic practice 120). The server 104 monitors such information and determines whether such measurement devices cause measurements to be inaccurate. For example, any time the server 104 identifies an inaccurate measurement, the server 104 may also increment a counter associated with the measurement device 102 that generated the inaccurate measurement. The server 104 may periodically compare a value indicated by the counter to a threshold equipment error. Should the counter value exceed the threshold equipment error, the server 104 may generate an equipment error flag for that measurement device 102. The equipment error flag would, in such an example, be indicative of a pattern of inaccurate measurements and, thereby, a potential technical issue associated with the measurement device 102. For example, the equipment error flag may indicate to the ophthalmic practice 120 that the measurement device 102 should be evaluated or recalibrated to ensure proper operation, as described above.

FIG. 3 below describes a sequence diagram in which the processor 124 of measurement device 102 verifies measurements generated by the measurement device 102. While the sequence diagram 300 and the corresponding description refer to components of the system 100 of FIG. 1, the steps of the sequence diagram 300 are not limited to that example embodiment and may apply to various other combinations of components. Furthermore, the sequence diagram 300 is not required to perform each of or only the shown steps and is not limited to performing the indicated steps in any particular order.

As described above, the sequence diagram 200 illustrates the operations of a cloud-based server 104 for obtaining and verifying measurements generated by the measurement device 102. On the other hand, the sequence diagram 300 illustrates the operations of the processor 124 of the measurement device 102 for obtaining and verifying measurements generated by the device features 123 of the measurement device 102 in a similar manner as the sequence diagram 200. The sequence diagram 300 depicts interactions between the processor 124, the device features 123, and the user interface 128 of the measurement device 102 and the data store 106. The sequence diagram 300 performs many operations that are similar to the operations shown in the sequence diagram 200 of FIG. 2. Corresponding steps between the sequence diagrams 200 and 300 have corresponding functionality and operations, and so forth. Thus, for steps in the sequence diagram 300 that correspond to steps in the sequence diagram 200, corresponding description will not be duplicated for brevity.

In the sequence diagram 300, communication steps 302-310 correspond to communication steps 202-210, with the patient identification data being received by the user interface 128 and communicated to the processor 124. The processor 124 requests and receives the patient profile 115 from the data store 106 before providing the patient profile 115 (at least partly) to the user interface 128. In certain embodiments, the communication between processor 124 or measurement device 102 and data store 106 may be performed directly or indirectly (e.g., through a server).

At communication step 312, the processor 124 may request that the device features 123 generate measurements for the anatomical characteristic of the patient's eye 110.

At processing step 314, the device features 123 generate the measurements.

Steps 316-323 correspond to steps 214-221 of the sequence diagram 200. Note that at step 318, in some embodiments, after determining that the measurement is inaccurate, the processor 124 may automatically cause the device features 123 to re-measure the patient's eye without any input from the medical practitioner.

### Example Communication Flow to Rank Ophthalmic practices

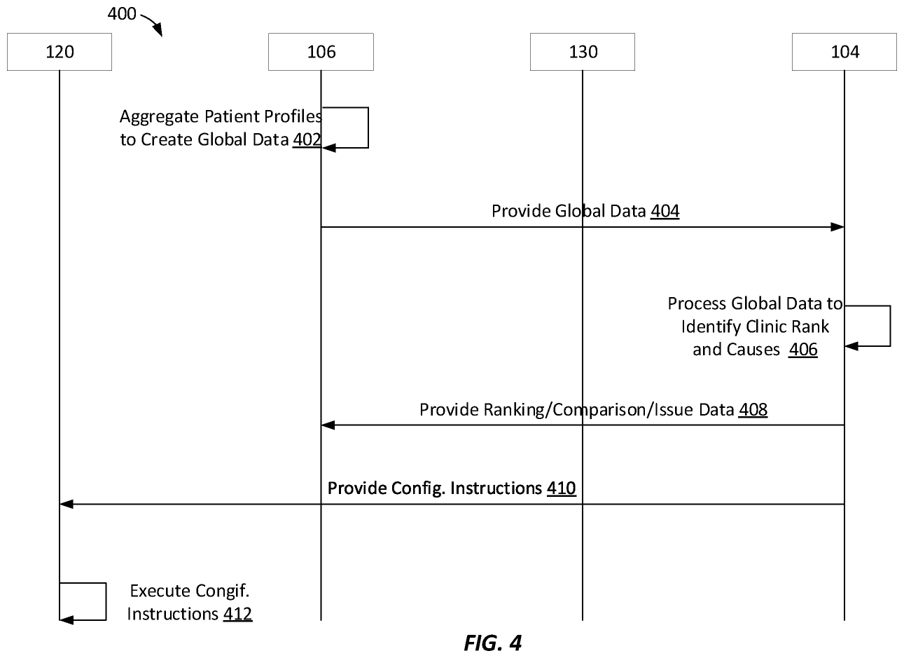

In some embodiments, the data store 106 aggregates information from a plurality of ophthalmic practices, for example, according to a geographic region. The data store 106 aggregates data from the patient profiles 115 stored in the data store 106. By aggregating such data, the server 104 can generate ranking information or recommendations for the multiple ophthalmic practices, as described in further reference to FIG. 4.

FIG. 4 is a sequence diagram 400 illustrating communications exchanged between or processing performed by components of, for example, the system 100 of FIG. 1 to aggregate information from a plurality of ophthalmic practices and generate ranking information based thereon. The ranking information, when provided to the individual ophthalmic practices, may enable the ophthalmic practices to improve measurements and procedures and, therefore, patient outcomes. In some embodiments, the flow diagram 400 includes processing that provides low ranked ophthalmic practices with recommendations or suggestions to improve the ophthalmic practices' measurements, satisfactory surgery results, and ranking.

While the sequence diagram 400 and corresponding description include reference to components of the system 100, the steps of the sequence diagram 400 are not limited to that example embodiment and may similarly apply to various other combinations of components and/or use cases. Furthermore, the sequence diagram 400 is not required to perform each of or only the shown steps and is not limited to performing the indicated steps in the shown order.

The sequence diagram 400, as shown, begins with processing step 402, where a data store 106 aggregates patient profiles 115 for patients that interacted with the ophthalmic practice 120 or the peer ophthalmic practices 130 to create a global data set. In some embodiments, aggregating the patient profiles 115 comprises formatting the aggregated patient profiles. Formatting may comprise ensuring that the patient profiles include the same fields (for example, the patient's historical and demographic information 116, optical measurements 117, actual treatment data 118 associated with the patient's surgery, and patient satisfaction information 119 for the corresponding patient, as introduced above).

At communication step 404, the data store 106 provides the global data set to the server 104 or provides the server 104 with access to the global data set.

At processing step 406, the server 104 processes the global data set to compare data between different ophthalmic practices to generate ranking information for the ophthalmic practices. In some embodiments, the server 104 analyzes the global data to rank ophthalmic practices based on the number of positive refractive outcomes for patients, where positive refractive outcomes are identified based on the patient satisfaction information 119. In some embodiments, the ranking for individual ophthalmic practices corresponds to or represents a quality score for the individual ophthalmic practices.

In some cases, the patient satisfaction information 119 alone may not provide the whole picture regarding a quality of the ophthalmic practice. In some embodiments, the server 104 incorporates analysis of the pre- and intraoperative measurements with the patient satisfaction information 119 when ranking the ophthalmic practices. For example, the server 104 may rank the ophthalmic practice with the highest number of satisfied patients (e.g., patients with positive refractive outcomes) and the highest number of accurate pre- and intra-operative measurements.

In some embodiments, the server 104 may rank ophthalmic practices based on the difference between left and right eye measurements (e.g., axial length measurements, average keratometry measurements, etc.) for the patients of each practice. For example, a ranking may be generated for each practice based on the formula below.

Ranking=(Σ(ALOD-ALOS)ₙ/AAL)+Σ(AKOD-AKOS)ₙ/AK))/NPP

In the formula above, ALOD refers to the axial length of the left eye for patient n. ALOD refers to the axial length of the right eye for patient n. AAL refers to the average axial length measured at the practice. AKOD refers to the average keratometry of the left eye for patient n. AKOS refers to the average keratometry of the right eye for patient n. AK refers to the average keratometry measured at the practice. NPP refers to the number of patients n.

In some embodiments, the server 104 may analyze an ophthalmic practice's ranking and optical measurements, actual treatment data, and patient satisfaction information for the corresponding patients of the ophthalmic practice to generate recommendations to improve the ophthalmic practice's ranking, measurement accuracies, and/or patient satisfaction.

For example, the server 104 may determine, based on the patient profiles 115 of patients from a particular ophthalmic practice having a low rank, that the cause of many inaccurate measurements is a certain medical practitioner at the ophthalmic practice who consistently provides inaccurate measurements and overrides re-measurement requests. The server 104 may identify the medical practitioner based on continuously analyzing patient profiles 115 associated with a certain ophthalmic practice. For example, the information included in the analysis may comprise the number of inaccurate measurements, the number of re-measurement request overrides, the number of patients who reported poor outcomes, the number of poor refractive outcomes (e.g., as indicated by post-operative measurements), etc. By analyzing this information for all patients and comparing different medical practitioners at the ophthalmic medical practice based on these parameters, the server 104 is able to determine that the cause of the low rank for the ophthalmic practice is the medical practitioner's inaccurate measurements.

In certain embodiments, the server 104 may determine, based on the patient profiles 115 of patients from a particular ophthalmic practice, that the cause of the inaccurate measurements that resulted in the ophthalmic practice's low rank is technical issues associated with a certain measurement device. For example, in some embodiments, the measurement device 102 may be out of calibration, be broken, or have other issues, which can cause inaccurate measurements. For example, the server 104 may continuously analyze the number of inaccurate measurements, the number of re-measurement request overrides, patients who reported poor outcomes, the number of poor refractive outcomes (e.g., as indicated by post-operative measurements), and other information to determine that all or most of the practice's poor patient outcomes are due to inaccurate measurements provided by a certain measurement device.

In some embodiments, the server 104 may provide recommendations to rectify the causes for the low ranking and, thus, improve the ophthalmic practice rank. For example, where the server 104 determines that the measurement device 102 causes the inaccurate measurements, the server 104 may recommend recalibration, replacement, or other actions to cure the issues. Similarly, where the server 104 determines that a medical practitioner causes the inaccurate measurements, the server 104 may identify the medical practitioner and recommend training or other actions to cure the issues.

In some embodiments, the server 104 identifies proposed device parameter settings for the measurement device 102 to improve measurements based on the patient specific measurement criteria and/or based on identified causes for inaccurate measurements. In some embodiments, the server 104 provides the proposed settings to the ophthalmic practice having the low rank, where the proposed settings are obtained from an ophthalmic practice with a higher rank.

At communication step 408, the server 104 provides the identified ranking information, causes for poor ranking or other issues, and recommendations to cure issues to the data store 106. In some embodiments, though not shown, the server 104 provides this information directly to the associated ophthalmic practices, such as the ophthalmic practice 120. In some embodiments, the server 104 provides the ranking information for the multiple ophthalmic practices to the data store 106 and separately sends the identified causes and/or solutions to specific, impacted ophthalmic practices as a separate communication (not shown). In certain embodiments, the server 104 may transmit configuration instructions (e.g., software patch, software update, calibration instructions, and the like) to resolve an issue that has been identified with, for example, measurement device 102. Communication step 410 illustrates an example of this transmission.

At communication step 410, the server 104 transmits configuration instructions to measurement device 102 at the ophthalmic practice 120 to automatically reconfigure (e.g., recalibrate, update, and/or change the settings of the measurements device 102).

At processing step 412, the measurement device 102 receives and executes the configuration instructions, which would cause the measurement device 102 to automatically change its configuration.

### Example Processing Systems

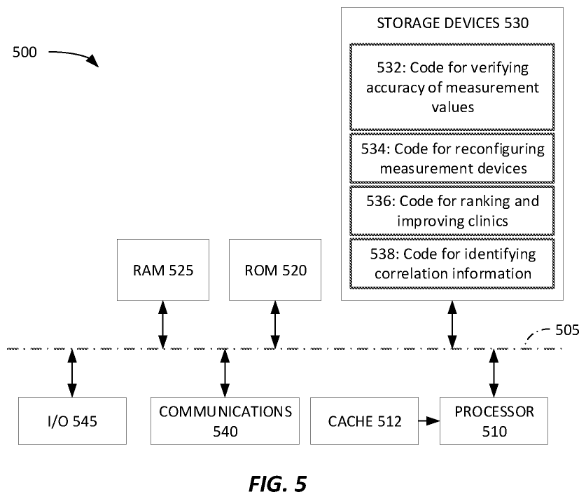

FIG. 5 is a diagram of an embodiment of a computing system 500 that may be representative of one or more of the measurement device 102, the server 104, and the like. Specifically, the computing system 500 may be configured to perform operations illustrated in one or more of the sequence diagrams 200, 300, and 400 and operations 600.

FIG. 5 illustrates computing system 500 where the components of the system 500 are in electronic communication with each other, for example, via a system bus 505. The bus 505 couples a processor 510 to various memory components, such as a read only memory (ROM) 520, a random access memory (RAM) 525, and the like (e.g., PROM, EPROM, FLASH-EPROM, and/or any other memory chip or cartridge). The system 500 may further include a cache 512 of high-speed memory connected to, in close proximity to, or integrated with the processor 510. In some embodiments, the system 500 may access data stored in the ROM 520, the RAM 525, and/or one or more storage devices 530 through the cache 512 for high-speed access by the processor 510.

In some embodiments, the one or more storage devices 530 store software modules, such as software modules 532, 534, 536, 538, and the like. When executed by the processor, the software modules 532, 534, 536, and 538 cause the processor 510 to perform various operations, such as the processes described herein. In some embodiments, one or more of the software modules 532, 534, 536, or 538 includes the ML models or other algorithms described herein.

The software module 532 comprises instructions (for example, in the form of computer-readable code) that program the processor 510 to verify the accuracy of measurements using the measurement criteria described above. The software module 534 comprises instructions that program the processor 510 to reconfigure measurement devices using configuration instructions, as described above. The software module 536 comprises instructions that program the processor 510 to generate ranking information for the ophthalmic practices, as described above. The software module 538 comprises instructions that program the processor 510 to determine patient-specific measurement criteria, such as threshold distances (e.g., using ML models or libraries).

Although the system 500 is shown with only one processor 510, the processor 510 may be representative of one or more central processing units (CPUs), multi-core processors, microprocessors, microcontrollers, digital signal processors (DSPs), field programmable gate arrays (FPGAs), application specific integrated circuits (ASICs), graphics processing units (GPUs), tensor processing units (TPUs), and the like. In some examples, the system 500 may be implemented as a stand-alone subsystem, as a board added to a computing device, as a virtual machine, or as a cloud-based processing machine.

To enable user interaction with the system 500 or communications between systems, the system 500 includes a communication interface 540 and input/output (I/O) devices 545. In some examples, the communication interfaces 540 includes one or more network interfaces, network interface cards, and the like to provide communication according to one or more network or communication bus standards. In some examples, the communication interface 540 includes an interface for communicating with the system 500 via a network. In some examples, the I/O devices 545 may include on or more user interface devices (e.g., graphical user interfaces (e.g., user interface 128), keyboards, pointing/selection devices (e.g., mice, touch pads, scroll wheels, track balls, touch screens, and/or the like), audio devices (e.g., microphones and/or speakers), sensors, actuators, display devices, and the like).

Each of the one or more storage devices 530 may include non-transitory and non-volatile storage such as that provided by a hard disk, an optical medium, a solid-state drive, and the like. In some examples, each of the one or more storage devices 530 is co-located with the system 500 (for example, a local storage device) or remote from the system 500 (for example, a cloud storage device).

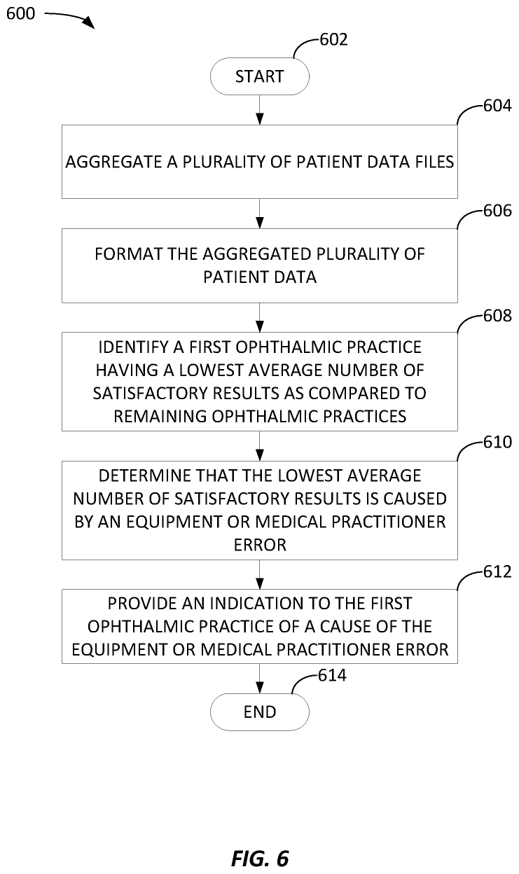

FIG. 6 depicts example operations 600 for aggregating information from a plurality of ophthalmic practices and identifying one or more causes for poor refractive outcomes associated with an ophthalmic practice according to embodiments of the present disclosure. For example, operations 600 may be performed by one or more components of the system 100 FIG. 1, such as the server 104.

At block 604, a plurality of patient profiles, such as patient profiles 115, are received and/or aggregated. The aggregated patient profiles may be stored as the global data introduced above.

At block 606, the aggregated plurality of patient profiles is formatted.

At block 608, the lowest ranked ophthalmic practice is identified based on having the lowest average number of satisfied patients or results as compared to remaining ophthalmic practices of the plurality of ophthalmic practices.

At block 610, the cause of the lowest average number of positive results being an equipment or medical practitioner error is determined.

At block 612, the system provides an indication to the first ophthalmic practice of a cause of the equipment or medical practitioner error.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. In addition, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein might be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. In addition, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An ophthalmic measurement device, comprising:
one or more ophthalmic measurement features configured to generate a measurement for an anatomical characteristic of an eye of a patient;
a user interface configured to enable a medical practitioner to interact with the ophthalmic measurement device;
a memory; and
a hardware processor in data communication with the memory and configured to:
determine whether the measurement satisfies measurement criteria based on comparing the measurement with the measurement criteria;
upon determining that the measurement does not satisfy the measurement criteria, cause the one or more ophthalmic measurement features to generate a new measurement for the anatomical characteristic;
determine whether the new measurement satisfies the measurement criteria based on comparing the new measurement with the measurement criteria; and
upon determining that the new measurement satisfies the measurement criteria, cause the user interface to display the new measurement.

2. The ophthalmic measurement device of claim 1, wherein upon determining that the measurement does not satisfy the measurement criteria, the hardware processor further:
causes the user interface to display a prompt to remeasure the anatomical characteristic; and
receive user input to remeasure the anatomical characteristic in response to the prompt, wherein causing the one or more ophthalmic measurement features to generate the new measurement is in response to the user input.

3. The ophthalmic measurement device of claim 1, wherein:
upon determining that the measurement does not satisfy the measurement criteria, the hardware processor is further configured to:
analyze the measurement and the measurement criteria to identify one or more proposed device parameter settings for the one or more ophthalmic measurement features for improving the measurement; and
cause the user interface to display the one or more proposed device parameter settings.

4. The ophthalmic measurement device of claim 3, wherein the hardware processor is further configured to automatically reconfigure the one or more ophthalmic measurement features based on the one or more proposed device parameter settings and a confirmation of the one or more proposed device parameters settings received through user input.

5. The ophthalmic measurement device of claim 1, wherein:
the measurement criteria comprises another measurement for the anatomical characteristic of another eye of the patient;
comparing the measurement with the measurement criteria comprises determining if a difference between the measurement and the other measurement falls within a threshold distance; and
determining that the measurement does not satisfy the measurement criteria comprises determining that the difference between the measurement and the other measurement does not fall within the threshold distance.

6. The ophthalmic measurement device of claim 5, wherein the threshold distance is patient specific.

7. The ophthalmic measurement device of claim 6, wherein the hardware processor is further configured to:
determine the threshold distance based on a patient profile of the patient, the patient profile including demographic information for the patient and fields for storing at least one of pre-operative measurements, intra-operative measurements, post-operative measurements, actual treatment data, or satisfaction information for the patient.

8. The ophthalmic measurement device of claim 1, wherein:
the measurement criteria comprises a threshold range of expected values within which the measurement is expected to fall; and
determining that the measurement does not satisfy the measurement criteria comprises determining that the measurement does not fall within the range of expected values.

9. The ophthalmic measurement device of claim 8, wherein the hardware processor is further configured to determine that the one or more ophthalmic measurement features require calibration, reconfiguration, or maintenance based on a determination that the measurement does not fall within the range of expected values.

10. The ophthalmic measurement device of claim 1, wherein the ophthalmic measurement device comprises one or more of a keratometer, an optical biometry device, an autorefractometer, a corneal topographer, an ocular wavefront aberrometer, an optical coherence tomography (OCT) device, or an ophthalmometer.

11. The ophthalmic measurement device of claim 1, wherein:
the measurement criteria comprises a previously generated measurement for the anatomical characteristic of the eye;
comparing the measurement with the measurement criteria comprises determining if a difference between the measurement and the previously generated measurement falls within a threshold distance; and
determining that the measurement does not satisfy the measurement criteria comprises determining that the difference between the measurement and the previously generated measurement does not fall within the threshold distance.

12. An ophthalmic measurement system, comprising:
an ophthalmic measurement device configured to generate a measurement for an anatomical characteristic of an eye of a patient;
a user interface configured to enable a medical practitioner to interact with the ophthalmic measurement device;
a hardware processor communicatively coupled to the ophthalmic measurement device and configured to:
determine whether the measurement satisfies measurement criteria based on comparing the measurement with the measurement criteria;
upon determining that the measurement does not satisfy the measurement criteria, cause the ophthalmic measurement device to generate a new measurement for the anatomical characteristic;
determine whether the new measurement satisfies the measurement criteria based on comparing the new measurement with the measurement criteria; and
upon determining that the new measurement satisfies the measurement criteria, cause the user interface to display the new measurement.

13. The ophthalmic measurement system of claim 12, wherein upon determining that the measurement does not satisfy the measurement criteria, the hardware processor further:
causes the user interface to display a prompt to remeasure the anatomical characteristic; and
receive user input to remeasure the anatomical characteristic in response to the prompt, wherein causing the ophthalmic measurement device to generate the new measurement is in response to the user input.

14. The ophthalmic measurement system of claim 12, wherein:
upon determining that the measurement does not satisfy the measurement criteria, the hardware processor is further configured to:
analyze the measurement and the measurement criteria to identify one or more proposed device parameter settings for the ophthalmic measurement device for improving the measurement; and
cause the user interface to display the one or more proposed device parameter settings.

15. The ophthalmic measurement system of claim 14, wherein the hardware processor is further configured to automatically reconfigure the ophthalmic measurement device based on the one or more proposed device parameter settings and a confirmation of the one or more proposed device parameters settings received through user input.

16. The ophthalmic measurement system of claim 12, wherein:
the measurement criteria comprises another measurement for the anatomical characteristic of another eye of the patient;
comparing the measurement with the measurement criteria comprises determining if a difference between the measurement and the other measurement falls within a threshold distance; and
determining that the measurement does not satisfy the measurement criteria comprises determining that the difference between the measurement and the other measurement does not fall within the threshold distance.

17. The ophthalmic measurement system of claim 16, wherein the threshold distance is patient specific.

18. The ophthalmic measurement system of claim 17, wherein the hardware processor is further configured to:
determine the threshold distance based on a patient profile of the patient, the patient profile including demographic information for the patient and fields for storing at least one of pre-operative measurements, intra-operative measurements, post-operative measurements, actual treatment data, or satisfaction information for the patient.

19. The ophthalmic measurement system of claim 12, wherein:
the measurement criteria comprises a threshold range of expected values within which the measurement is expected to fall; and
determining that the measurement does not satisfy the measurement criteria comprises determining that the measurement does not fall within the range of expected values.

20. A method for reconfiguring an ophthalmic measurement device, the method comprising:
aggregating a plurality of patient profiles to form a global dataset, each patient profile associated with a corresponding patient treated at one of a plurality of ophthalmic practices and comprising one or more of measurements of an anatomical characteristic of a patient's eye, procedure results, or demographics and patient history information for the corresponding patient;
formatting each patient profile into a common format;
identifying a first ophthalmic practice of the plurality of ophthalmic practices having a lowest average number of satisfactory results as compared to remaining ophthalmic practices of the plurality of ophthalmic practices;
determining that the lowest average number of satisfactory results for the first ophthalmic practice is caused by an error associated with the ophthalmic measurement device; and
automatically reconfiguring the ophthalmic measurement device.
